SEQUENCE LISTING (34 sequences)

SEQ ID NO: 1 - DNA, 26 bp, Artificial sequence
  Synthetic primer
ccgggcatatgatgactgggggtgag

SEQ ID NO: 2 - DNA, 29 bp, Artificial sequence
  Synthetic primer
gggaattcactagttcagaggttgatgtc

SEQ ID NO: 3 - DNA, 28 bp, Artificial sequence
  Synthetic primer
tggtcatatgatgaccaccgacgccgcg

SEQ ID NO: 4 - DNA, 32 bp, Artificial sequence
  Synthetic primer
gggaattcactagttcactgcaaccaggcttc

SEQ ID NO: 5 - DNA, 30 bp, Artificial sequence
  Synthetic primer
ggccgctagcatgatcttccttgtgggact

SEQ ID NO: 6 - DNA, 36 bp, Artificial sequence
  Synthetic primer
gggaattcccactagttcatacgacttccagtcggg

SEQ ID NO: 7 - DNA, 32 bp, Artificial sequence
  Synthetic primer
ccgggcatatgatgaatgggatcagtgattcc

SEQ ID NO: 8 - DNA, 32 bp, Artificial sequence
  Synthetic primer
gggaattcactagtctagtgctcctcggtggg

SEQ ID NO: 9 - DNA, 30 bp, Artificial sequence
  Synthetic primer
gggccatatgatgcgggtactgctgacgtc

SEQ ID NO: 10 - DNA, 29 bp, Artificial sequence
  Synthetic primer
gggaattcactagtctagccggcgtggcg

SEQ ID NO: 11 - DNA, 28 bp, Artificial sequence
  Synthetic primer
ccgggcatatgatgggtgatcggaccgg

SEQ ID NO: 12 - DNA, 29 bp, Artificial sequence
  Synthetic primer
gggaattcactagttcatccggcggtcct

SEQ ID NO: 13 - DNA, 27 bp, Artificial sequence
  Synthetic primer
ctggcatatggtggcgggcggtttcga

SEQ ID NO: 14 - DNA, 29 bp, Artificial sequence
  Synthetic primer
gggaattcactagttcacctgccggtgct

SEQ ID NO: 15 - DNA, 30 bp, Artificial sequence
  Synthetic primer
ccgggcatatgatgagcagttcggacgagc

SEQ ID NO: 16 - DNA, 29 bp, Artificial sequence
  Synthetic primer
gggaattcactagtctaccgctgcccggg

SEQ ID NO: 17 - DNA, 27 bp, Artificial sequence
  Synthetic primer
ccgggcatatgatggacgtccccttcc

SEQ ID NO: 18 - DNA, 32 bp, Artificial sequence
  Synthetic primer
gggaattcactagttcaagccccagccttgag

SEQ ID NO: 19 - DNA, 30 bp, Artificial sequence
  Synthetic primer
cctagcatatgatgaccacgaccgatcgcg

SEQ ID NO: 20 - DNA, 32 bp, Artificial sequence
  Synthetic primer
gggaattcactagttcagagctcgacggggca

SEQ ID NO: 21 - DNA, 30 bp, Artificial sequence
  Synthetic primer
cggccatatgatgcgcgtcgtcttctcctc

SEQ ID NO: 22 - DNA, 32 bp, Artificial sequence
  Synthetic primer
gggaattcactagttcatcgtggttctctcct

SEQ ID NO: 23 - DNA, 26 bp, Artificial sequence
  Synthetic primer
ccgggcatatgatgaaacgcgcgctg

SEQ ID NO: 24 - DNA, 29 bp, Artificial sequence
  Synthetic primer
gggaattcactagttcacgaaccgttgcg

SEQ ID NO: 25 - DNA, 32 bp, Artificial sequence
  Synthetic primer
ccgggcatatgatgaacacaactcgtacggca

SEQ ID NO: 26 - DNA, 29 bp, Artificial sequence
  Synthetic primer
gggaattcactagttcaccttccgcgcag

SEQ ID NO: 27 - DNA, 29 bp, Artificial sequence
  Synthetic primer
ccgggcatatgatgtacgagggcgggttc

SEQ ID NO: 28 - DNA, 29 bp, Artificial sequence
  Synthetic primer
gggaattcactagttcatccgcgcacacc

SEQ ID NO: 29 - DNA, 28 bp, Artificial sequence
  Synthetic primer
ccggtcatatgatgacgaccgttcccga

SEQ ID NO: 30 - DNA, 31 bp, Artificial sequence
  Synthetic primer
ggcggaattcactagttcatccgtcgagccg

SEQ ID NO: 31 - DNA, 29 bp, Artificial sequence
  Synthetic primer
ccggtcatatggagcacaagaggaaccga

SEQ ID NO: 32 - DNA, 31 bp, Artificial sequence
  Synthetic primer
ggcggaattcactagtggtcaccgaggtggc

SEQ ID NO: 33 - DNA, 28 bp, Artificial sequence
  Synthetic primer
ccggtcatatgttgaccaccatcgacga

SEQ ID NO: 34 - DNA, 31 bp, Artificial sequence
  Synthetic primer
ggcggaattcactagtctacgccgactgcct

(19) United States
(12) Patent Application Publication
(10) Pub. No.: US 2011/0262971 A1
(43) Pub. Date: Oct. 27, 2011

(54) Genetically Modified E. coli Strains for Producing Erythromycin

(75) Inventors: Blaine Pfeifer, Somerville, MA (US); Haoran Zhang, Medford, MA (US)

(73) Assignee: Tufts University, Boston, MA (US)

(21) Appl. No.: 12/764,230

(22) Filed: Apr. 21, 2010

(51) Int. Cl. C12P 19/62; C12N 1/21

(52) U.S. Cl. 435/76; 435/252.33

(57) ABSTRACT

Genetically modified E. coli strains capable of producing erythromycins, particularly erythromycin A.

### STATEMENT AS TO FEDERALLY SPONSORED RESEARCH

This invention was made with government support under #1R43AI74224-01 & #0712019 awarded by the NIH and NSF, respectively. The government has certain rights in the invention.

### BACKGROUND OF THE INVENTION

Erythromycins, produced by Saccharopolyspora erythraea, are a family of macrolide antibiotics effective against a broad spectrum of bacterial pathogens. Erythromycin biosynthesis involves two complicated pathways, i.e., (1) the polyketide synthesis pathway for producing 6-deoxyerythronolide B (6dEB), the macrocyclic core of erythromycin, and (2) the sugar synthesis and attachment pathway for synthesizing the erythromycin sugar moieties and attaching them to the macrocyclic core.

Typically, production of erythromycin was built around large-scale Saccharopolyspora erythraea fermentation. The slow growth kinetics of this organism in addition to rudimentary molecular biology protocols emphasizes the opportunity to establish production in a technically convenient and more engineering-friendly heterologous host system. The goal of such an effort would be to produce erythromycin A, the most therapeutically potent erythromycin species. Due to the structural complexity, chemical synthesis of erythromycins is technically difficult and economically infeasible.

It is of great interest to develop a cost-effective production process for obtaining erythromycins in large quantity.

### SUMMARY OF THE INVENTION

One aspect of this invention features a genetically modified E. coli strain for producing erythromycin (e.g., erythromycin A). This E. coli strain contains genes involved in the erythromycin sugar synthesis and attachment pathway, including an eryF gene, an eryBV gene, an eryCIII gene, a first gene set including an eryBVI gene, an eryBVII gene, an eryBII gene, an eryBIII gene, and an eryBIV gene, and a second gene set including an eryCI gene, an eryCII gene, an eryCIV gene, an eryCV gene, and an eryCVI gene. Preferably, it further contains an eryG gene, an eryK gene, an ermE gene, or a combination thereof. In one example, the E. coli strain of this invention contains two expression operons, one including the eryBII, eryBIII, eryBIV, eryBV, eryBVI, eryBVII, and ermE genes and the other including the eryCI, eryCII, eryCIII, eryCIV, eryCV, eryCVI, eryF, eryG, and eryK genes.

The E. coli strain described above can further contain genes involved in the polyketide synthesis pathway, including a DEBS1 gene, a DEBS2 gene, and a DEBS3 gene. Optionally, it can further contain an sfp gene, an accA1 or accA2 gene, a pccB gene, a birA gene, a prpE gene or a combination thereof.

Each of the aforementioned genes is in operative linkage to an E. coli promoter for expression in E. coli cells. When necessary, it can be fused with a nucleotide sequence coding for a protein tag, e.g., the hexa-His tag.

To improve expression of any of the genes listed above, one or more genes coding for chaperone proteins can be introduced into the above-described E. coli strain. Suitable chaperone proteins include, but are not limited to, dnaK, dnaJ, grpE, groES, groEL, and tig.

Also within the scope of this invention is a method for obtaining erythromycin from any of the genetically modified E. coli strains of this invention. This method includes (i) cultivating one of the E. coli strains in a medium under conditions (e.g., 22-30° C.) suitable for expression of the aforementioned genes and production of erythromycin and (ii) collecting the medium for isolation of the erythromycin. In one example, the medium contains a 6-deoxyerythronolide B substrate (e.g., 6-deoxyerythronolide B), which can be produced by an E. coli strain carrying the genes involved in the polyketide biosynthesis pathway.

The details of one or more embodiments of the present invention are set forth in the description below. Other features or advantages of the present invention will be apparent from the following drawings and detailed description of several examples, and also from the appended claims.

### BRIEF DESCRIPTION OF THE DRAWINGS

The drawings are first described.

FIG. 1 is a scheme illustrating the erythromycin biosynthesis pathway and the enzymes involved in it.

FIG. 2 is a diagram showing the structures of expression constructs pHZT1 and pHZT2.

FIG. 3 is a chart showing the levels of erythromycin A, erythromycin B, erythromycin C, and erythromycin D produced by E. coli strain STAR (DE3 carrying expression plasmids pHZT1, pHZT2, and pGro7).

FIG. 4 is a chart showing the levels of erythromycins in three genetically modified E. coli strains.

FIG. 5 is a chart showing the levels of erythromycins and erythromycin analogs produced by a genetically modified erythromycin-producing E. coli strain in the presence of 6dEB-CH2.

### DETAILED DESCRIPTION OF THE INVENTION

Described herein are genetically modified E. coli strains capable of producing an erythromycin, e.g., erythromycin A, B, C, D, or an analog thereof.

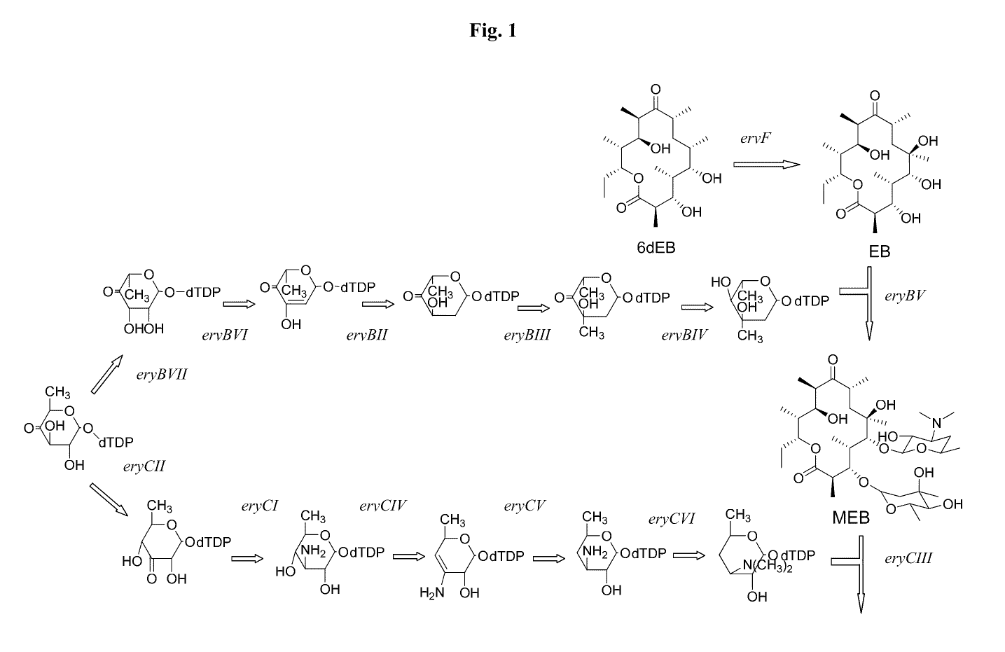

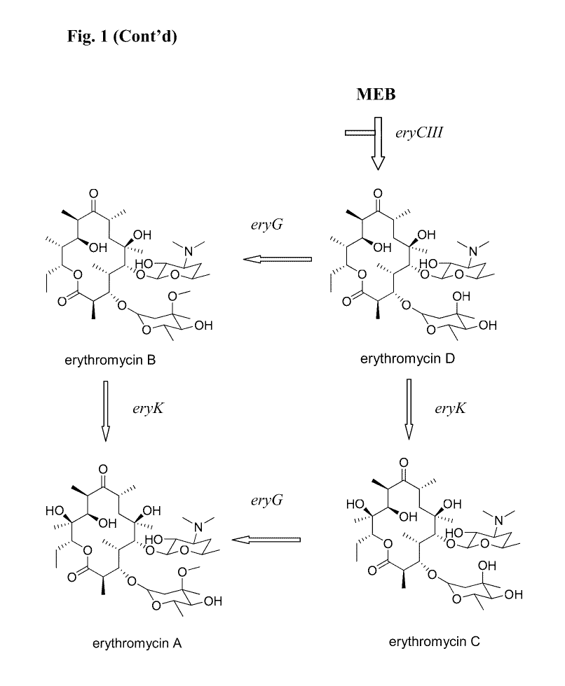

This E. coli strain can carry S. erythraea genes involved in the erythromycin sugar synthesis and attachment pathway described in FIG. 1, including eryF, eryBV, eryCIII, eryBVI, eryBVII, eryBII, eryBIII, eryBIV, eryCI, eryCII, eryCIV, eryCV, eryCVI, and optionally, one or both of eryG and eryK. Preferably, it also carries ermE, an erythromycin resistance gene. An S. erythraea gene is a gene found in any strain of S. erythraea, e.g., strain NRRL 2338, the genome of which is disclosed in GenBank Accession Number NC 009142 (17 MAR. 2010). Table 1 below lists the S. erythraea genes mentioned above isolated from S. erythraea NRRL 2338 and the encoded enzymes:

TABLE 1
S. erythraea Genes Involved in Erythromycin
Biosynthesis and Resistance

| Gene | GenBank accession number | Enzyme Encoded |
|---|---|---|
| eryBII | U77454 (822597 to 823598 | TDP-4-keto-6-deoxyhexose |
| | in NC_009142) | 2,3-reductase |
| eryBIII | 826478 to 827710 in NC_009142 | Deoxyhoxose |
| | | methyltransferase |
| eryBIV | U77459 (786751 to 787719 | dTDP-4-keto-6-deoxy-L- |
| | in NC_009142) | hexose 4-reductase |
| eryBV | 785504 to 786751 in NC_009142 | L-mycarosyltransferase |
| eryBVI | U77459 (783121 to 784584 | NDP-4-keto-6-deoxy- |
| | in NC_009142) | glucose 2,3-dehydratase |
| eryBVII | U77459 (779802 to 780404 | Deoxyhexose epimerase |
| | in NC_009142) | |
| eryCI | 831725 to 832825 in NC_009142 | Deoxyhexose |
| | | aminotransferase |
| eryCII | U77454 (820239 to 821324 | Deoxyhexose isomerase |
| | in NC_009142) | |
| eryCIII | 821335 to 822600 in NC_009142 | L-desosaminyltransferase |
| eryCIV | U77459 (781919 to 783124 | NDP-6-deoxyhexose |
| | in NC_009142) | 3,4-dehydratase |
| eryCV | U77459 (780412 to 781881 | NDP-4,6-dideoxyhexose |
| | in NC_009142) | 3,4-enoyl reductase |
| eryCVI | U77459 (784738 to 785451 | Deoxyhexose N- |
| | in NC_009142) | methyltransferase |
| eryF | 825267 to 826481 in NC_009142 | 6dEB hydroxylase |
| eryG | 823630 to 824550 in NC_009142 | O-methyltransferase |
| eryK | 778654 to 779847 in NC_009142 | Erythromycin C-12 |
| | | hydroxylase |
| ermE | X51891 (830393 to 831538 | Dimethyladenosine |
| | in NC_009142) | transferase |

Counterpart genes from other S. erythraea strains can be retrieved from GenBank or isolated from a target strain via conventional methods. Both native genes and their degenerative variants can be used to construct the modified E. coli strains. In one example, a native S. erythraea gene is subjected to codon optimization by introducing E. coli preferred codons.

To prepare the E. coli strains, the S. erythraea genes mentioned above can be isolated from their natural source(s) and cloned into one or more expression cassettes, in which each of the genes is in operative linkage to an E. coli promoter. A promoter sequence is a nucleotide sequence containing elements that initiates the transcription of an operably linked nucleic acid sequence. At a minimum, a promoter contains an RNA polymerase binding site. It can further contain one or more enhancer elements which, by definition, enhances transcription, or one or more regulatory elements that control the on/off status of the promoter. An E. coli promoter is a promoter that functions within E. coli. Representative E. coli promoters include the β-lactamase and lactose promoter systems (see Chang et al., Nature 275:615-624, 1978), the SP6, T3, T5, and T7 RNA polymerase promoters (Studier et al., Meth. Enzymol. 185:60-89, 1990), the lambda promoter (Elvin et al., Gene 87:123-126, 1990), the trp promoter (Nichols and Yanofsky, Meth. in Enzymology 101:155-164, 1983), the tac and trc promoters (Russell et al., Gene 20:231-243, 1982), and pCold (see U.S. Pat. No. 6,479,260).

The expression cassette(s) mentioned above, included in one or more plasmids, can be introduced into an E. coli host stain, such as JM109, BL21(DE3), DH5α, and MC1061, via conventional recombinant technology. The resultant positive transformants can be examined to determine their expression of the S. erythraea genes by, e.g., Western blot, SDS-PAGE, or enzymatic activity analysis. In one example, the expression cassette(s) is incorporated into the chromosome of the host strain via homologous recombination. In another example, it is extra-chromosomal.

To improve expression of the S. erythraea genes, one or more of them can be fused with a nucleotide sequence coding for a protein tag, preferably a hexa-His tag. Alternatively or in addition, the E. coli strains described above can be further transformed by one or more plasmids for expression of one or more chaperone proteins. E. coli chaperone proteins are well known in the art. Examples include, but are not limited to, dnaK, dnaJ, grpE, groES, groEL, and tig.

Any of the E. coli strains described above can be cultured in a medium in the presence of a 6dEB substrate under suitable conditions (e.g., at 22° C.) to produce erythromycin. The term “6-dEB substrate” used herein refers to 6dEB and its analogs, i.e., compounds having the same macrocylic core as 6dEB (see FIG. 1) with one or more of the side groups in 6dEB (except the two hydroxyl groups for sugar attachment) replaced with a suitable group(s), e.g., alkyl group, an alkenyl group, a ketone group, a hydroxyl group, alkyl, benzyl, or other starter groups, and any combination of chirality associated with the previously mentioned groups (where applicable). A 6dEB substrate can be prepared either by chemical synthesis or by a genetically modified microorganism (e.g., E. coli) designed for producing such (see Pfeifer et al., Science 291:1790-1792 (2001). When a 6dEB analog is used, the E. coli strains of this invention produce its corresponding erythromycin analog that has the same replacement side group(s) linked to the macrocylic core.

After being cultivated for a suitable period (e.g., 3-5 days), the culture medium is collected and erythromycin contained in it is isolated via routine methods, e.g., chromatography.

Any of the genetically modified E. coli stains described above can further contain genes involved in the polyketide synthesis pathway for producing 6dEB, i.e., the genes coding for deoxyerythronolide B synthase (DEBS). DEBS is a megadalton α₂β₂γ₂ complex containing α, β, and γ subunits, which are encoded by DEBS1, DEBS2, and DEBS3 genes, respectively. See Pfeifer et al., Science 291:1790-1792, 2001. In addition, the E. coli strain can contain one or more of the genes coding for phosphopantetheinyl transferase, propionyl-CoA synthetase, acyl-CoA carboxylase complex A subunit, propionyl-CoA carboxylase complex B subunit, and carboxylase holoenzyme synthetase. Such genes can be obtained from a suitable microorganism, e.g., E. coli, S. erythraea, S. coelicolor, and B. subtilis. Table 2 below lists examples of the just-mentioned genes, their GenBank accession numbers, and the enzymes they encode.

TABLE 2
Genes Coding for Enzymes Involved in 6dEB Synthesis

| Gene | GenBank Accession Number | Enzyme Encoded |
|---|---|---|
| DEBS1 | 787944 to 798581 in NC_009142 | Erythromycin polyketide synthase |
| | | modules 1 and 2″ |
| DEBS2 | 800023 to 810726 in NC_009142 | Erythromycin polyketide synthase |
| | | modules 3 and 4″ |
| DEBS3 | 810727 to 820242 in NC_009142 | Erythromycin polyketide synthase |
| | | modules 5 and 6″ |
| sfp | X65610 (01, OCT. 1992) | Phosphopantetheinyl transferase |
| accA1 or | AF113603 (accA1) or AF113604 | acyl-CoA carboxylase complex A |
| accA2 | (accA2) (08, DEC. 1999) | subunit |
| pccB | AF113605 (08, DEC. 1999) | propionyl-CoA carboxylase |
| | | complex B subunit |
| birA | 4270802 to 4271767 in CP000948 | bifunctional biotin-acetylCoA |
| | (05, JUN. 2008) | carboxylase holoenzyme |
| | | synthetase and DNA-binding |
| | | transcriptional repressor |
| prpE | 3458163 to 3460049 in NC 012947 | Propionyl-CoA synthetase |
| | (06, JUL. 2009) | |

Referring to the genes listed in Table 2, both native genes and their functional variants can be used to construct the E. coli strain of this invention. Functional variants include degenerative variants of their native counterparts (e.g., those produced by codon optimization) and nucleotide sequences coding for functional equivalents of the proteins encoded by the native genes. A functional equivalent of a protein refers to a polypeptide that shares at least 85% (e.g., 90%, 95%, or 99%) sequence identity to the protein and has the same bioactivity as the protein.

The percent identity of two amino acid sequences is determined using the algorithm of Karlin and Altschul Proc. Natl. Acad. Sci. USA 87:2264-68, 1990, as modified in Karlin and Altschul Proc. Natl. Acad. Sci. USA 90:5873-77, 1993. Such an algorithm is incorporated into the BLASTN and BLASTX programs (version 2.0) of Altschul et al., J. Mol. Biol. 215:403-10, 1990. BLAST protein searches can be performed with the BLASTX program, score=50, wordlength=3 to obtain amino acid sequences homologous to the protein molecules of the invention. Where gaps exist between two sequences, Gapped BLAST can be utilized as described in Altschul et al., Nucleic Acids Res. 25:3389-3402, 1997. When utilizing BLAST and Gapped BLAST programs, the default parameters of the respective programs (e.g., BLASTX and BLASTN) can be used.

The just-described E. coli strain, carrying genes involved in both the polyketide synthesis pathway and the sugar synthesis and attachment pathway, can be used to produce erythromycin in the absence of 6dEB.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference.

### Example 1

### Production of Erythromycin in Genetically Modified E. coli Strains

### Materials and Methods

### (i) Reagents and Chemicals

The reagents and chemicals used in this study were purchased from Fisher Scientific (Pittsburgh, Pa., USA) or Sigma (St. Louis, Mo., USA). All restriction enzymes and the Phusion High-Fidelity PCR Master Mix were purchased from New England Biolabs (Ipswich, Mass., USA). PCR primers were synthesized by Operon (Huntsville, Ala., USA).

### (ii) Culture Medium and Cell Growth

LB medium was used during molecular biology protocols and gene expression experiments. Production medium was used during erythromycin production experiments. One liter of production medium contained 5 g yeast extract, 10 g tryptone, 15 g glycerol, 10 g sodium chloride, 3 mL 50% v/v antifoam B, 100 mM 4-(2-Hydroxyethyl)-1-piperazineethanesulfonic acid (HEPES) buffer, and was adjusted to pH 7.6 by NaOH before use.

E. coli Genehogs, a DH10B™-derived E. coli strain, was used as the host cell in all gene cloning efforts except for plasmid eryK-pCDF construction, which was carried out in E. coli JM109. E. coli BL21(DE3) was used as the host cell for gene expression.

All E. coli strains were grown at 22, 30, or 37° C. and 250 rpm. The cell growth of erythromycin-producing strains in production medium was monitored by recording OD₆₀₀ values versus time.

(iii) Gene Isolation, Expression, and Plasmid Construction

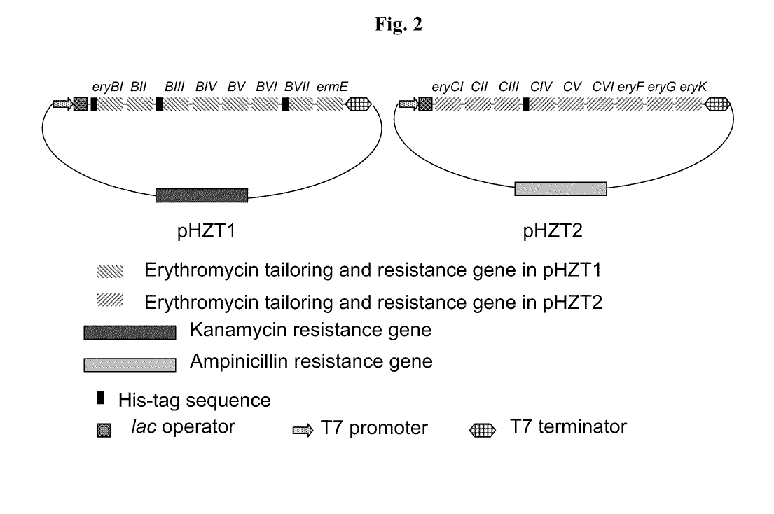

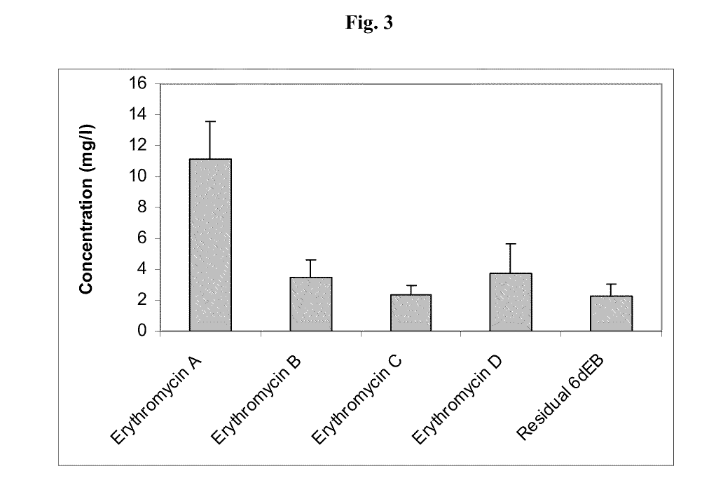

Restriction enzyme digestions, SDS-PAGE, and other standard molecular biology techniques were performed as described by Sambrook et al. (1989). Erythromycin tailoring and resistance genes (see FIG. 2) were PCR amplified from genomic DNA isolated from S. erythraea using the primers listed in Table 3 below:

TABLE 3
Primers for Amplifying Genes Involved in Erythromycin Biosynthesis

| Gene | Oligonucleotides sequence |
|---|---|
| BI | Forward 5′-CCGGGCATATG ATGACTGGGGGTGAG-3′ (SEQ ID NO: 1) |
| | Reverse 5′-GGGAATTCACTAGTTCAGAGGTTGATGTC-3′ (SEQ ID NO: 2) |
| BII | Forward 5′-TGGTCATATG ATGACCACCGACGCCGCG-3′ (SEQ ID NO: 3) |
| | Reverse 5′-GGGAATTCACTAGTTCACTGCAACCAGGCTTC-3′ (SEQ ID NO: 4) |
| BIII | Forward 5′-GGCCGCTAGCATGATCTTCCTTGTGGGACT-3′ (SEQ ID NO: 5) |
| | Reverse 5′-GGGAATTCCCACTAGTTCATACGACTTCCAGTCGGG-3′ (SEQ ID NO: 6) |
| BIV | Forward 5′-CCGGGCATATG ATGAATGGGATCAGTGATTCC-3′ (SEQ ID NO: 7) |
| | Reverse 5′-GGGAATTCACTAGTCTAGTGCTCCTCGGTGGG-3′ (SEQ ID NO: 8) |
| BV | Forward 5′-GGGCCATATGATGCGGGTACTGCTGACGTC-3′ (SEQ ID NO: 9) |
| | Reverse 5′-GGGAATTCACTAGTCTAGCCGGCGTGGCG-3′ (SEQ ID NO: 10) |
| BVI | Forward 5′-CCGGGCATATG ATGGGTGATCGGACCGG-3′ (SEQ ID NO: 11) |
| | Reverse 5′-GGGAATTCACTAGTTCATCCGGCGGTCCT-3′ (SEQ ID NO: 12) |
| BVII | Forward 5′-CTGGCATATG GTGGCGGGCGGTTTCGA-3′ (SEQ ID NO: 13) |
| | Reverse 5′-GGGAATTCACTAGTTCACCTGCCGGTGCT-3′ (SEQ ID NO: 14) |
| ermE | Forward 5′-CCGGGCATATG ATGAGCAGTTCGGACGAGC-3′ (SEQ ID NO: 15) |
| | Reverse 5′-GGGAATTCACTAGTCTACCGCTGCCCGGG-3′ (SEQ ID NO: 16) |
| CI | Forward 5′-CCGGGCATATG ATG GACGTCCCCTTCC-3′ (SEQ ID NO: 17) |
| | Reverse 5′-GGGAATTCACTAGTTCAAGCCCCAGCCTTGAG-3′ (SEQ ID NO: 18) |
| CII | Forward 5′-CCTAGCATATG ATGACCACGACCGATCGCG-3′ (SEQ ID NO: 19) |
| | Reverse 5′-GGGAATTCACTAGTTCAGAGCTCGACGGGGCA-3′ (SEQ ID NO: 20) |
| CIII | Forward 5′-CGGCCATATGATGCGCGTCGTCTTCTCCTC-3′ (SEQ ID NO: 21) |
| | Reverse 5′-GGGAATTCACTAGTT CATCGTGGTTCTCTCCT-3′ (SEQ ID NO: 22) |
| CIV | Forward 5′-CCGGGCATATG ATGAAACGCGCGCTG-3′ (SEQ ID NO: 23) |
| | Reverse 5′-GGGAATTCACTAGTTCACGAACCGTTGCG-3′ (SEQ ID NO: 24) |
| CV | Forward 5′-CCGGGCATATG ATGAACACAACTCGTACGGCA-3′ (SEQ ID NO: 25) |
| | Reverse 5′-GGGAATTCACTAGTTCACCTTCCGCGCAG-3′ (SEQ ID NO: 26) |
| CVI | Forward 5′-CCGGGCATATG ATGTACGAGGGCGGGTTC-3′ (SEQ ID NO: 27) |
| | Reverse 5′-GGGAATTCACTAGTTCATCCGCGCACACC-3′ (SEQ ID NO: 28) |
| eryF | Forward 5′-CCGGTCATATG ATGACGACCGTTCCCGA-3′ (SEQ ID NO: 29) |
| | Reverse 5′-GGCGGAATTCACTAGTTCATCCGTCGAGCCG-3′ (SEQ ID NO: 30) |
| eryG | Forward 5′-CCGGTCATATG GAGCACAAGAGGAACCGA-3′ (SEQ ID NO: 31) |
| | Reverse 5′-GGCGGAATTCACTAGTGGTCACCGAGGTGGC-3′ (SEQ ID NO: 32) |
| eryK | Forward 5′-CCGGTCATATG TTGACCACCATCGACGA-3′ (SEQ ID NO: 33) |
| | Reverse 5′-GGCGGAATTCACTAGTCTACGCCGACTGCCT-3′ (SEQ ID NO: 34) |

Initially, all PCR fragments were cloned into pET21c. The eryBIII PCR product was inserted using the NheI and EcoRI restriction sites, the eryBV PCR product was inserted using the NdeI and Sad restriction sites, and the eryCIII PCR product was inserted using the NdeI and HindIII restriction sites. The PCR products for the other genes were inserted using the NdeI and EcoRI restriction sites. The resultant plasmids were introduced into E. coli BL21(DE3) and positive transformants were selected via routine procedures. All PCR products were verified by DNA sequencing at the Tufts University Core Facility.

E. coli BL21(DE3) cultures (1 mL) for each cloned gene were grown at 37° C., induced with 100 mM isopropyl β-D-1-thiogalactopyranoside (IPTG) at an OD₆₀₀ of value of 0.6, and cultured overnight. Cellular proteins were then analyzed by SDS-PAGE to determine expression of the cloned genes. Those genes that did not demonstrate expression using pET21c (i.e., BI, BIII, BVII, and CIV) were transferred to pET28a (containing an N-terminal hexa-his tag) by NheI/HindIII (for eryBIII) or NdeI/HindIII (for eryBI, BVII, and CIV) and their expressions were tested again following the same procedures described above.

Two operons containing the erythromycin tailoring and resistance genes were constructed as follows. eryBI, BII, BIII, BIV, BV, BVI, BVII and ermE genes were cloned sequentially via a XbaI/SpeI and Sad digestion and ligation strategy. The resulting plasmid pHZT1 was a derivative of pET28 (Kan^{R}). The same strategy was used to complete a second operon containing eryCI, CII, CIII, CIV, CV, CVI, eryF, eryG, and eryK. Here, genes were inserted into pET21 (Carb^{R}) using sequential XbaI/SpeI and HindIII digestions resulting in plasmid pHZT2. See FIG. 2. Genes for operon construction were taken from the individual pET21 expression plasmids except for BI, BIII, BVII, and CIV, which were taken from the pET28 constructs. The alignment of the erythromycin tailoring and resistance genes in both pHZT1 and pHZT2 is shown in FIG. 2.

To construct a separate plasmid carrying eryK, the eryK gene was PCR amplified, digested with NdeI and XhoI, and ligated into pCDFDuet-1 (streptomycin resistant) treated with the same enzymes to produce plasmid pHZT4.

Chloramphenicol-resistant chaperone plasmids (Takara) pG-KJE8, pGro7, pKJE7, pG-Tf2, and pTf16, provided by Takaba Bio Inc., were utilized to improve erythromycin production.

### (iv) Production Cultures and LC-MS Analysis

Plasmids pHZT1 and pHZT2, in combination with one or more of the chaperone plasmids and eryK-pCDF, were transformed into BL21(DE3) and STAR(DE3). Starter cultures were grown in LB medium at 22° C. overnight and used to inoculate 1 and 10 ml production cultures containing appropriate antibiotics, 0.1 mM IPTG, and 2 mg/ml arabinose (when needed). After culturing for 2 days at 22° C., 6dEB was added to the cultures at a final concentration of 50 mg/L. Erythromycin production was analyzed by MS analysis after continued culturing for an additional 3 days.

For MS analysis, roxithromycin (Sigma) was used as an internal standard. One milliliter of the production cultures was extracted with 0.5 ml ethyl acetate. After centrifugation at 12,000 rpm for 1 min, the extract was transferred to an eppendorf tube and air-dried. The dried extract was then resuspended in 50 μl of methanol containing 2.5 mg/L roxithromycin.

For quantitative measurement, a LC-MS calibration curve was prepared using commercially available erythromycin A as a standard. Specifically, known amounts of erythromycin A were added to E. coli cultures (without the needed plasmids) grown under the same condition as the erythromycin-producing cultures. The standard samples were extracted and resuspended as described above and subjected to LC-MS analysis. The samples were analyzed using an LTQ XL Linear Ion Trap Mass Spectrometer (Thermo Electron Corporation) coupled with a Finnigan Surveyor LC system (Thermo Electron Corporation). A linear gradient of 100% water to 100% acentonitrile over 15 minutes was applied to all samples. The ratio of the standard erythromycin A peak areas to the roxithromycin internal standard was correlated to experimental erythromycin concentrations by using a calibration curve made before every experimental analysis.

### (v) Preparation of Robust Erythromycin Producing Strains

In order to improve erythromycin production, E. coli production strains were subjected to increasing concentrations of exogenous erythromycin A. Specifically, BL21(DE3)/pHZT1/pHZT2/pGro7 and STAR(DE3)/pHZT1/pHZT2/pGro7 were grown in LB medium at 37° C. overnight, diluted 4×10⁴ fold with fresh LB medium, and 300 μl of each of the diluted culture were spread onto an LB agar plate containing 100 mg/l ampicillin, 50 mg/l kanamycin, 20 mg/l chloramphenicol, 0.1 mM IPTG, 2 mg/ml arabinose and erythromycin ranging from 10-500 mg/l. The colonies that survive on the high erythromycin concentration plates were picked, transformed with plasmid eryK-pCDF, and positive transformants were selected and cultured for erythromycin production.

### (vi) Bioassay Study

Erythromycins in the culture medium were extracted using ethyl acetate, dried, and then dissolved in methanol. Certain amount of the resulting erythromycin-methanol solution, after normalization, was added onto the top of a filter paper disk placed onto an growth medium plate prepared by mixing 20 ul B. subtilis overnight culture with 20 ml warm LB agar. The positive control used was the external erythromycin standard added into overnight production medium culture of BL21(DE3) and extracted by the method described above.

### Results

### (i) Protein Analysis

Via SDS-PAGE analysis, expression of his-eryBI, his-BIII, his-BVII and his-CIV fusion proteins and expression of the other 12 erythromycin tailoring genes were observed. The enzymatic activities of certain gene products were confirmed in functionality tests. For example, the introduction of the plasmid carrying ermE gene conferred the host cell resistance to erythromycin up to 400 mg/l. Similarly, a 6dEB derivative, erythronolide B, was produced by the E. coli strain carrying the eryF gene, indicating that the product of this gene (6dEB hydroxylase) was functional.

### (ii) Production of Erythromycin B and D

Plasmids pHZT1 and pHZT2 described above were introduced into E. coli BL21(DE3) and STAR(DE3); positive transformants were grown under 22° C. and 37° C. in production medium for 5 days (2 days prior to and 3 days after 6dEB addition). LC-MS results indicate that the cultures contained erythronolide B (EB), which was converted from exogenous 6dEB by eryF.

The E. coli strains BL21(DE3, pHZT1, 2) and STAR(DE3, pHZT1, 2) were further transformed with chaperone plasmids pG-KJE8, pGro7, pKJE7, pG-Tf2, pTf16, or a combination thereof. The resulting strains were grown in the production medium and induced by IPTG and arabinose. LC-MS analysis showed that erythromycin B and D were detected in both BL21(DE3) carrying pHZT1, pHZT2, pG-KLE8, and pGro7 and STAR(DE3) carrying the same plasmids. Further investigation demonstrated that the strains with pGro7 produced more erythromycin B and D than the strains with pG-KJE8. Therefore, pGro7 was used in all the following erythromycin heterologous biosynthesis studies.

(iii) Production of Erythromycin A and C

The erythromycin-producing strains mentioned above were further transformed with plasmid pHTZ4, carrying the eryK gene. Production of erythromycin A was observed in E. coli strains BL21 (DE3)/pHZT1/pHZT2/pHZT4/pGro7 and STAR(DE3)/pHZT1/pHZT2/pHZT4/pGro7. Erythromycin C, derived from erythromycin D by eryK, was also found to be produced in these E. coli strains. LC-MS analysis also showed that EB, MEB, erythromycin B, C, and D, along with a large amount of unused 6dEB, were in the cultures. Furthermore, it was found that BL21 (DE3)/pHZT1/pHZT2/pHZT4/pGro7 produced more erythromycin than STAR(DE3)/pHZT1/pHZT2/pHZT4/pGro7.

### (iv) Improved Erythromycin A Production

To screen for erythromycin resistant clones, E. coli strains BL21(DE3)/ermE-pET21, BL21(DE3)/pHZT1/pHZT2/pGro7, and STAR(DE3)/pHZT1/pHZT2/pGro7 were grown on a plate containing different concentrations of erythromycin A. Clones surviving at 300 mg/l or 500 mg/l erythromycin were selected for analysis.

The following three BL21(DE3)/pHZT1/pHZT2/pGro7 clones were further investigated in this study for erythromycin production:

Clone 1: before erythromycin A selection,

Clone 2: resistant to 300 mg/l erythromycin A, and

Clone 3: resistant to 500 mg/l erythromycin A.

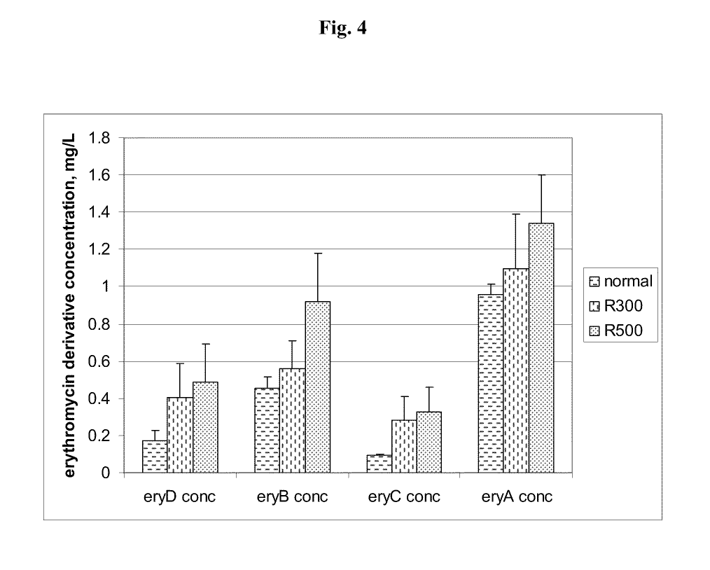

These three clones were first transformed with pHZT4 (carrying eryK). The resulting strains were grown at 22° C. for 5 days (2 days prior to and 3 days after 6dEB addition) and the production of erythromycin was analyzed by LC-MS. As shown in FIG. 4, clone 2 transformed with pHZT4 (designated “R300”) produced much more erythromycin A than clone 1 transformed with pHZT4 (designated “normal”) and the production of erythromycin A, B, C and D was dramatically increased in clone 3 transformed with pHZT4 (designated “R500”). 6dEB was also converted to the intermediates EB and MEB, which were both observed in LC-MS analyses.

### Example 2

### Production of Erythromycin-CH2 Analog in Genetically Modified E. coli Strains

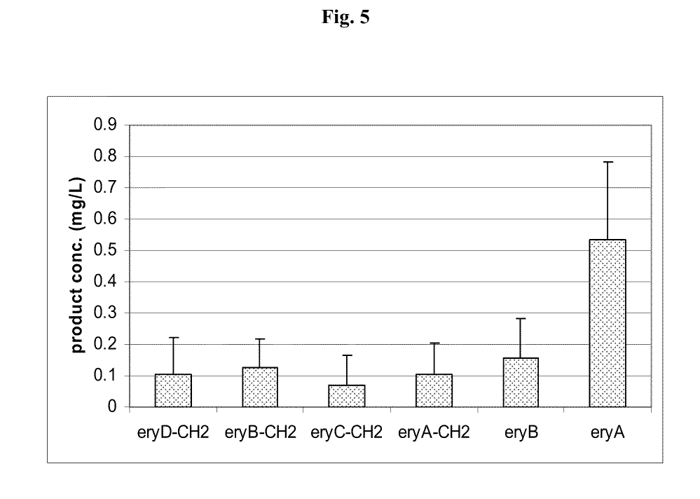

E. coli strain BL21(DE3)/pHZT1/pHZT2/pGro7 was cultured in the production medium described above supplemented with 6dEB-CH2 (13 mg/l) at 22° C. for 3 days. The culture medium was collected and subjected to LC-MS analysis to determine presence of erythromycin and its —CH2 analog in the culture. As shown in FIG. 5, both erythromycins (e.g., B and A) and their —CH2 analogs (i.e., eryD-CH2, eryB-CH2, eryC-CH2, and eryA-CH2) were found in the medium. These results indicate that when fed with 6dEB analogs, the genetically modified E. coli strains are capable of producing erythromycin analogs.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A genetically modified E. coli strain for producing erythromycin, comprising:
an eryF gene,
an eryBV gene,
an eryCIII gene,
a first gene set including an eryBVI gene, an eryBVII gene, an eryBII gene, an eryBIII gene, and an eryBIV gene, and
a second gene set including an eryCI gene, an eryCII gene, an eryCIV gene, an eryCV gene, and an eryCVI gene.

2. The genetically modified E. coli strain of claim 1, further comprising an eryG gene, an eryK gene, or both.

3. The genetically modified E. coli strain of claim 2, wherein the E. coli strain contains both the eryG gene and the eryK gene.

4. The genetically modified E. coli strain of claim 2, further comprising an ermE gene.

5. The genetically modified E. coli strain of claim 1, further comprising one or more exogenous genes each coding for a chaperone protein.

6. The genetically modified E. coli strain of claim 5, wherein the chaperone protein is selected from the group consisting of dnaK, dnaJ, grpE, groES, groEL, and tig.

7. The genetically modified E. coli strain of claim 2, further comprising one or more exogenous genes each coding for a chaperone protein.

8. The genetically modified E. coli strain of claim 7, wherein the chaperone protein is selected from the group consisting of dnaK, dnaJ, grpE, groES, groEL, and tig.

9. The genetically modified E. coli strain of claim 7, further comprising an ermE gene, wherein the one or more exogenous genes are a groES gene and a groEL gene, the eryBII, eryBIII, eryBIV, eryBV, eryBVI, eryBVII, and ermE genes are constructed in a first expression operon, the eryCI, eryCII, eryCIII, eryCIV, eryCV, eryCVI, eryF, eryG, and eryK genes are constructed in a second expression operon, and the groES and groEL genes are constructed in a third expression operon.

10. The genetically modified E. coli strain of claim 2, wherein one or more of the eryBII, eryBIII, eryBIV, eryBV, eryBVI, eryBVII, ermE, eryCI, eryCII, eryCIII, eryCIV, eryCV, eryCVI, eryF, eryG, and eryK genes are each linked with a nucleotide sequence coding for a protein tag.

11. The genetically modified E. coli strain of claim 7, wherein the protein tag is hexa-His.

12. The genetically modified E. coli strain of claim 1, further comprising a DEBS1 gene, a DEBS2 gene, and a DEBS3 gene.

13. The genetically modified E. coli strain of claim 12, further comprising an sfp gene.

14. The genetically modified E. coli strain of claim 12, further comprising an accA1 or accA2 gene, and a pccB gene.

15. The genetically modified E. coli strain of claim 14, further comprising a birA gene, a prpE gene, or a combination thereof.

16. The genetically modified E. coli strain of claim 14, further comprising an eryG gene and an eryK gene.

17. The genetically modified E. coli strain of claim 16, further comprising one or more exogenous genes each coding for a chaperone protein selected from the group consisting of dnaK, dnaJ, grpE, groES, groEL, and tig.

18. The genetically modified E. coli strain of claim 16, further comprising an ermE gene.

19. The genetically modified E. coli strain of claim 16, wherein one or more of the eryBII, eryBIII, eryBIV, eryBV, eryBVI, eryBVII, ermE, eryCI, eryCII, eryCIII, eryCIV, eryCV, eryCVI, eryF, eryG, and eryK genes are each linked with a nucleotide sequence coding for a hexa-His tag.

20. A method for producing erythromycin in E. coli, comprising
providing the genetically modified E. coli strain of claim 1,
cultivating the E. coli strain in a medium under conditions allowing production of the erythromycin, and
collecting the medium for isolation of the erythromycin.

21. The method of claim 20, wherein the genetically modified E. coli strain further comprises an eryG gene, an eryK gene, or both.

22. The method of claim 21, wherein the genetically modified E. coli strain further comprises an ermE gene.

23. The method of claim 21, wherein the medium contains a 6-deoxyerythronolide B substrate.

24. The method of claim 23, wherein the substrate is 6-deoxyerythronolide B.

25. The method of claim 24, wherein the 6-deoxyerythronolide B is produced in a genetically engineered E. coli strain carrying a DEBS1 gene, a DEBS2 gene, and a DEBS3 gene.

26. The method of claim 25, wherein the genetically engineered E. coli strain further carries an sfp gene, a birA gene, a preE gene, both a accA1 or accA2 gene, and a pccB gene, or a combination thereof.

27. The method of claim 20, wherein the genetically modified E. coli strain further comprises a DEBS1 gene, a DEBS2 gene, and a DEBS3 gene.

28. The method of claim 27, wherein the genetically modified E. coli strain further contains an sfp gene.

29. The method of claim 28, wherein the cultivating step is performed by culturing the E. coli strain at a temperature of 22-30° C.

30. The method of claim 27, wherein the genetically modified E. coli strain further comprises an accA1 or accA2 gene, and a pccB gene.

31. The method of claim 30, wherein the genetically modified E. coli strain further comprises a birA gene, a prpE gene, or a combination thereof.

32. The method of claim 31, wherein the genetically modified E. coli strain further comprises an eryG gene and an eryK gene.

33. The method of claim 32, wherein the genetically modified E. coli strain further comprises an ermE gene.
